(19) United States
(12) Patent Application Publication
(10) Pub. No.: US 2007/0275442 A1
(43) Pub. Date: Nov. 29, 2007

(54) Side Chain Deuterated Amino Acids Methods Of Use

(75) Inventors: Jonathan Miles Brown, Silver Spring, MD (US); Steven W. Homans, Bramhope (GB); Michael Chaykovsky, Columbia, MD (US); Jenny Hong Murray, Gaithersburg, MD (US)

Correspondence Address: ROTHWELL, FIGG, ERNST & MANBECK, P.C., 1425 K STREET, N.W., SUITE 800, WASHINGTON, DC, 20005, US

(73) Assignee: ProSpect Pharma, Columbia, MD (US)

(21) Appl. No.: 10/574,967

(22) PCT Filed: Oct. 7, 2004
(86) PCT No.: PCT/US04/32941

§ 371 Date: May 24, 2007

Related U.S. Application Data
(60) Provisional application No. 60/508,886, filed on Oct. 7, 2003.

(51) Int. Cl. G01N 33/53; C07C 211/01; G01N 24/08; C12P 21/06

(52) U.S. Cl. 435/71.1; 435/243; 436/86; 530/300; 562/553

(57) ABSTRACT

Protein structural determination using NMR techniques is improved through use of proteins in which one or more amino acids in the peptidic sequence are isotopically enriched in the sidechain with 2H and are isotopically enriched on the backbone with 13C, 15N, 2H or any combination thereof. This invention provides amino acids isotopically enriched as above, which can be used to synthesize isotopically labeled proteins and peptides for protein structural determinations by NMR, and methods for their synthesis. Other embodiments of the invention include peptidic molecules, media for peptidic molecule expression, methods of making isotopically labeled peptidic molecules and methods of determining structural information of a peptidic molecule.

This application claims benefit of prior co-pending U.S. provisional application Ser. No. 60/508,886, the disclosures of which are hereby incorporated by reference in their entirety.

### BACKGROUND OF THE INVENTION

1. Technical Field

This application relates to the field of drug design, and in particular to methods for obtaining high resolution NMR data for large protein systems such as membrane receptors and protein/protein complexes.

2. Description of the Background Art

Modern drug discovery research depends heavily on knowledge of the structures of biologically active macromolecules. This research would benefit substantially from enhancements in the capabilities and speed of three-dimensional structural analyses of proteins and other macromolecules, particularly valuable targets such as membrane receptors. Although approximately 50% of current drugs target membrane-bound proteins such G-Protein Coupled Receptors, at present virtually no structural information is available on this important class of molecules.

The past few years have seen rapid growth in methods for discovery and identification of candidate drugs. Genomic techniques, using rapid DNA sequencing methods and computer assisted homology identification, have enabled the rapid identification of target proteins as potential drug candidates. O'Brien, Nature, 385 (6616):472, 1997. Once identified, a target protein can be produced quickly using modern recombinant technology. Combinatorial chemistry, wherein large numbers of chemical compounds are simultaneously synthesized on plastic plates, frequently by robots, has revolutionized the synthesis of drug candidates, where libraries containing tens of thousands of compounds can be synthesized in a few months. See Gordon et al., J. Mol. Chem., 37(10):1385-1401, 1994. Each member of the library is then “screened” for binding to one or more target proteins. Compounds that bind are identified, and similar compounds are synthesized and screened. This process continues in an iterative manner until a drug candidate of suitably high binding affinity is identified.

Having information about the three-dimensional structure of a target protein allows one to design a “focused” combinatorial library, which mimics structures matching the potential binding region of the target protein, increasing the likelihood of finding potential drug candidates that interact with the biological molecule of interest. Further, having information about the three-dimensional structure of a protein/drug candidate complex can reveal additional details about how and where the binding occurs as well as strengths and weaknesses in the interaction and hence potential avenues for improving desired aspects of the binding interaction.

Unfortunately, using commonly available methods, while genomic techniques and combinatorial chemistry each are performed in months, methods to determine protein structure usually take much longer. Therefore, there is a need in the art for methods to increase the speed and accuracy of obtaining high resolution structures of proteins, including structures of proteins that are the targets of potential drug candidates.

X-ray crystallography is widely used to obtain an estimate of the structure of proteins and can provide the complete tertiary structure (global fold) of the backbone of a crystallized protein. This method, however, has several disadvantages. For example, only proteins which can be crystallized may be studied using X-ray crystallography. Some proteins, such as membrane proteins, are very difficult or impossible to crystallize. Moreover, crystallization of a protein can be very time-consuming and expensive. In addition, to obtain the structure of a protein/drug complex it is often necessary to prepare a second crystal using the protein and the drug, thus doubling the already difficult process of crystallization, a time-consuming task.

Another major disadvantage of X-ray crystallographic data is that the structural information obtained may be pertinent only to the crystalline structure of the protein and not to the structure of the protein in solution. Moreover, the bond angles present in a crystal structure may not be the same as those of the protein when it is in an active conformation and therefore may not provide information relevant to the biological or physiological system of interest, therefore providing misleading information about the structure of potential binding molecules.

Protein structure determination by high resolution Nuclear Magnetic Resonance (NMR) also is well known. In NMR spectroscopy, magnetization of certain atomic nuclei (usually protons) in a powerful magnetic field is detected by the absorption of radio waves. NMR has become a major tool in the study and analysis of small (<1 kD) molecules. To analyze larger molecules, such as proteins, it is essential in most applications to replace the natural abundance atoms of carbon and nitrogen (¹²C and ¹⁴N) universally with the NMR-active stable isotopes ¹³C and ¹⁵N to allow reliable assignment of each of the detected NMR signals. See Ikura et al., Biochemistry 29:4659-4667, 1990; Bax, Curr. Opin. Struct. Biol. 4:738-744, 1994. Using isotopic labeling of this type, NMR has allowed workers to determine the structure of several proteins. See Ikura et al., Biochemistry 30:9216-9228, 1991; Clore and Gronenborn, Nat. Struct. Biol. 4:849-853, 1997.

Early methods for determining protein structure using NMR used distance data derived from NOE (Nuclear Overhauser Effect) spectra. More recently, residual dipolar coupling measurements have become established as a method to obtain additional angular conformational restraints for determining the solution structures of proteins via high resolution multinuclear NMR. Tolman et al., Proc. Natl. Acad. Sci. USA 92:9270-9283, 1995; Tjandra et al., J. Am. Chem. Soc. 118:6264-6272, 1996; Tjandra and Bax, Science 278:1111-1114, 1997; Bax and Tjandra, J. Biomol. NMR 10:289-292. Methods for weak macromolecular alignment, such as lyotropic dilute liquid-crystalline solutions, have simplified measurement of these couplings for a variety of macromolecules. See Bax and Tjandra, J. Biomol. NMR 10:289-292, 1997; Losonczi et al., J. Biomol. NMR 12:447-451, 1998; Prosser et al., J. Am. Chem. Soc. 120:11010-11011, 1998; Clore et al., J. Am. Chem. Soc. 120:10571-10572, 1998; Hansen et al., Nat. Struct. Biol. 5:1065-1074, 1998; Kiddle and Homans, FEBS Lett. 436:128-130, 1998; Wang et al., J. Biomol. NMR 12:443-446, 1998; Ottinger and Bax, J. Biomol. NMR 13:187-191, 1999; Fleming et al., J. Am. Chem. Soc. 122:5224-5225, 2000; Rückert and Otting, J. Am. Chem. Soc. 122:7793-7797, 2000; Mueller et al., J. Mol. Biol. 300:197-212, 2000; Mueller et al., J. Biomol. NMR 18:183-188, 2000; Fowler et al., J. Mol. Biol. 304:447-460, 2000; Hus et al., J. Mol. Biol. 298:927-936, 2000; Hus et al., J. Am. Chem. Soc. 123:1541-1542, 2001.

In addition, Fesik et al. (Science 274:1531-34, 1996) have described a screening strategy in which libraries are screened using NMR. In this method, an isotopically labeled target protein is subjected to NMR in the absence and presence of drug candidate molecules and binding is detected from perturbations in the NMR spectrum. NMR also can be used to determine the structures of protein/ligand complexes in solution. See Shimizu et al.; J. Am. Chem. Soc., 121:5815-5816, 1999.

Isotopic substitution in a protein usually is accomplished by growing a bacterium or yeast, transformed by genetic engineering to produce the protein of choice, in a growth medium containing universally ¹³C—, ¹⁵N— and/or ²H-labeled substrates. Many such growth media are now commercially available. See, e.g., U.S. Pat. No. 5,324,658. In practice, bacterial growth media usually consist of ¹³C-labeled glucose and/or ¹⁵N-labeled ammonium salts dissolved in D₂O where necessary. Kay et al., Science 249:411, 1990 (and references therein); Bax, J. Am. Chem. Soc. 115:4369, 1993. Techniques for producing isotopically labeled proteins and other macromolecules, including glycoproteins, in mammalian or insect cells have also been described. See U.S. Pat. Nos. 5,393,669 and 5,627,044; Weller, Biochemistry 35:8815-23, 1996; Lustbader, J. Biomol. NMR 7:295-304, 1996.

In principle, NMR can provide structural data on drug targets such as a protein, unbound and/or complexed to a drug candidate. The actual use of NMR for these purposes, however, has been limited to the study of only relatively small proteins. Because the magnetization of the isotopically labeled nuclei in a protein (¹H, ¹³C, ¹⁵N) tends to diffuse more easily with increasing molecular weight, the signal-to-noise ratio decreases with the size of the molecule being studied, rendering the data more difficult to interpret as the protein size increases. In essence, this is because the very isotopes needed to assign the protein NMR signals in the first place, such as ¹³C, allow the magnetization to diffuse. In addition, the universal labeling yields split signals in the NMR spectrum. The signals being assigned are split into multiplets by neighboring isotopes, which results in both more and weaker signals. This splitting further degrades the signal with respect to noise. Taken together, these phenomena cause increasing overlap of signals and decreasing signal-to-noise ratio with increasing molecular weight, making determination of structure using these methods very laborious and time-consuming. Each of the split signals need to be assigned before structure determination can be commenced. Therefore, in practice, these methods can provide fairly accurate structures only of small and medium sized proteins. Structure determinations of proteins have been restricted to sizes of about 35 kD or less, and for the most part only to non-membrane proteins. Therefore, NMR has made only a modest impact to date on drug design.

Many attempts have been made to increase the sensitivity of NMR to increase the size of proteins that can be studied by the technique. Deuteration of ¹³C— and ¹⁵N-enriched protein leads to significant narrowing of the carbon 13 signals relative to protonated proteins, see Grzesiek and Bax, J. Am. Chem. Soc. 115:4369-4370, 1993, however, splitting of the signals from adjacent carbon atoms still occurs, lowering the signal-to-noise ratio and increasing the number of signals.

In another development, the introduction of TROSY spectroscopy, Pervushin et al., Proc. Natl. Acad. Sci. USA 94(23):12366-12371, 1997, has led to the observation of ¹⁵N resonances on very large molecular systems, provided that the molecule is fully deuterated. However, these ¹⁵N signals cannot be assigned without related carbon resonances and even with deuteration, carbon resonances disappear in universally ¹³C-labeled proteins at much smaller molecular sizes than ¹⁵N resonances accessed via TROSY methods. This method therefore does not increase the size of proteins that can be studied using NMR.

Recently, methods involving specific isotopic enrichment of the protein with ¹³C, ¹⁵N and ²H in the backbone only have overcome some of these problems and dramatically increased the resolution and sensitivity of NMR spectra in structural studies of some proteins. These methods facilitate detection and assignment of the NMR signals and the calculation of the structure of the protein. (Coughlin et al., J. Am. Chem. Soc. 121:11871-11874, 1999; Giesen et al., J. Biomol. NMR 19:255-260, 2001; U.S. Pat. Nos. 6,111,066 and 6,376,253. Specifically, splitting of the signals from adjacent carbon 13 atoms is removed by this method because side chain carbon 13 atoms are lacking. In consequence, each C-α carbon appears as a single signal, and if deuterated, with optimum intensity. In addition, because the carbon 13 atoms are linked to the nitrogen atoms in the backbone, the nitrogen signals also can be assigned. Therefore, all the signals from the backbone of the protein can be assigned.

These enhanced assigned signals then can be used to calculate the global fold of the protein using, inter alia, the measurement of dipolar couplings. Giesen et al., J. Biol. NMR 25:1-9, 2002. Further, the screening methods such as those described by Fesik et al. (Science 274:1531-1534, 1996), then can be performed using this structural information, greatly increasing the value of the screening.

Despite these advantages, however, the method described above for labeling a protein in the backbone only suffers from some serious drawbacks. First, although the method allows for deuteration at the C-α carbon, none of the other protons in the amino acid, i.e. in the sidechain, are deuterated. Therefore, proteins labeled in the backbone only according to U.S. Pat. Nos. 6,111,066 and 6,376,253 cannot be analyzed by TROSY-type spectrometry. Further, this lack of deuteration also causes higher rates of loss of magnetization in general with increasing molecular mass. Therefore, a need exists in the art for a method that overcomes these disadvantages.

### SUMMARY OF THE INVENTION

Accordingly, embodiments of this invention provide an amino acid wherein the sidechain of the amino acid is isotopically enriched with ²H and wherein the backbone of the amino acid is isotopically enriched with an isotope selected from the group consisting of ¹³C, ¹⁵N, ²H and any combination thereof, with the proviso that the amino acid is not isotopically enriched with ²H at every hydrogen. Further embodiments provide an amino acid as described above wherein the backbone of the amino acid is isotopically enriched with an isotope selected from the group consisting of ¹³C, ¹⁵N, ²H and any combination thereof. Additional further embodiments provide an amino acid as described above, wherein the α-carbon proton of the amino acid is isotopically enriched with ²H.

In yet further embodiments, the invention provides a method of synthesizing the amino acids described above which comprises obtaining glycine that optionally is isotopically enriched in the backbone with an isotope selected from the group consisting of ¹³C, ¹⁵N and ²H or any combination thereof; chemically derivatizing the glycine; adding a deuterated side chain to the chemically derivatized glycine in a stereo-selective manner to produce a protected sidechain deuterated amino acid; and deprotecting the sidechain deuterated amino acid. In yet a further embodiment, the invention provides a method of synthesizing the amino acids described above which comprises obtaining glycine that optionally is isotopically enriched in the backbone with an isotope selected from the group consisting of ¹³C, ¹⁵N and ²H or any combination thereof; chemically derivatizing the glycine; adding a deuterated side chain to the chemically derivatized glycine in a stereo-selective manner to produce a protected sidechain deuterated amino acid; deuterating the α-carbon of the protected sidechain deuterated amino acid; and deprotecting the sidechain deuterated amino acid.

In further embodiments, the invention provides peptidic molecules which comprise at least one amino acid as described above. In some embodiments, the peptide molecules comprise at least one species of amino acid wherein the side chain of each occurrence of said species of amino acid is isotopically enriched with ²H, wherein the backbone of each occurrence of said species of amino acid is isotopically enriched with an isotope selected from the group consisting of ¹³C, ¹⁵N, ²H and any combination thereof, or wherein the α-carbon proton of each occurrence of said species of amino acid is isotopically enriched with ²H.

In yet further embodiments, the invention provides media capable of supporting the growth of cells in culture which comprises at least one amino acid as described above.

In yet further embodiments, the invention provides methods of producing an isotopically labeled peptide molecule which comprise providing a medium as described above; providing a cell culture that expresses the peptide molecule; growing the cell culture in the medium under protein-producing conditions such that the cell expresses the peptide molecule in isotopically labeled form; and isolating the isotopically labeled peptide molecule from the medium.

In yet further embodiments, the invention provides methods of determining structural information for a peptidic molecule which comprise producing the peptidic molecule according to the method described above; and subjecting the peptidic molecule to nuclear magnetic resonance.

### BRIEF DESCRIPTION OF THE FIGURES

FIG. 1 shows a chemical synthetic scheme for isotopically labeled valine.

FIG. 2 shows a chemical synthetic scheme for a deuterated sidechain precursor for leucine.

### DETAILED DESCRIPTION OF THE PREFERRED EMBODIMENTS

Embodiments of the invention provide means for increasing the resolution and sensitivity of NMR spectra obtained from proteins, particularly large proteins such as membrane receptors, etc., and therefore allow more detailed information regarding protein structure more quickly and more accurately than previously possible. This improvement in NMR spectroscopic techniques involves (1) increasing the resolution and sensitivity of key signals in the NMR spectrum, (2) eliminating the splitting of the key signals by an adjacent NMR active nucleus and (3) isolating the NMR active nuclei required to obtain the desired information on protein global fold in an environment of NMR inactive nuclei. This is accomplished by specifically isotopically labeling at least one of the amino acids which make up the protein to be studied by NMR with any combination of ¹³C, ¹⁵N, and ²H in the backbone of the amino acid, with optional labeling of the α-carbon proton, and also with ²H in the side chain of the amino acid. This approach is a departure from current NMR labeling techniques, where the goal has been to prepare proteins either in a universally labeled form (with labeling at every position in the protein molecule) or labeled in the backbone of the amino acid chain only, avoiding side chain labeling.

Embodiments of the invention provide an amino acid that is isotopically enriched with an isotope selected from the group consisting of ¹³C, ¹⁵N, and ²H or any combination thereof in the backbone and that also is isotopically enriched with ²H in the sidechain. In other embodiments, the invention provides a method for synthesizing such amino acids which comprises (a) chemically derivatizing glycine and (b) adding a deuterated sidechain in a stereo-selective fashion. In other embodiments, the invention provides methods for synthesizing a deuterated sidechain of amino acids which comprise (a) deuteration of existing unlabeled sidechain precursors or (b) assembling appropriate sidechains in deuterated form.

Although the methods of this invention are suitable for the study by NMR of any peptidic molecule of three or more amino acids in length, and therefore encompasses both proteins and peptides, the description, for simplicity, will refer only to proteins. The discussion therefore applies to both peptides and proteins, even when the term protein is used. It is understood that the terms “protein” and “peptidic molecule” as used in this application, both refer to any peptide chain of three or greater amino acids, or, for example, peptides and proteins of any length. Preferably, the peptidic molecule is about 5 kD or greater molecular weight.

The compositions and methods of the present invention therefore advantageously may be employed in connection with proteins having molecular masses of about 5 kD or more, or proteins of about 50 amino acid residues or more. The methods are particularly useful for proteins of 20-30 kD or larger, which have been difficult to study using prior art methods, and even more particularly proteins of 50 or 55 kD or more or 75 kD, or proteins of 100 kD or longer. Therefore any protein of 50, 60, 70, 80, 90, 100, 110, 120, 130, 140, 150 kD or more, or complexes of such proteins, are suitable for structural and dynamic information determinations according to embodiments of this invention. The methods may be used to study membrane proteins as well. Of course, smaller proteins and peptides may be studied using the inventive methods, including oligopeptides and any peptide of three or greater amino acids.

Proteins containing the specifically labeled amino acids may be chemically synthesized from scratch or expressed by cells in culture, for example by bacterial, yeast, mammalian or insect cells.

Amino acids have been chemically synthesized in unlabeled forms by various means. Some have been synthesized in specifically isotopically labeled forms (see, for example, Martin, Isotopes Environ. Health Stud., 32:15, 1996; Schmidt, Isotopes Environ. Health Stud., 31:161, 1995). Ragnarsson et al. (J. Chem. Soc. Perkin Trans. 1:2503, 1994) synthesized 1,2-¹³C₂, ¹⁵N Ala, Phe, Leu, Tyr; 1,2-¹³C₂, 3′,3′,3′-²H₃, ¹⁵N Ala; 1,2-¹³C₂, 3′,3′-²H₂, ¹⁵N Phe and 3′,3′,3′-²H₃Ala. Ragnarsson et al. also synthesized 1,2-¹³C₂, 2-²H, ¹⁵N Ala, Leu and Phe and 1,2-¹³C₂, 2,2-²H₂, ¹⁵N Gly, which were used partly for conformational studies of a pentapeptide, Leu-enkephalin. Unkefer synthesized ¹⁵N labeled Ala, Val, Leu, Phe as well as 1-¹³C, ¹⁵N Val. Other methods have been described in Duthaler, Tetrahedron 50:1539, 1994; Schöllkopf, Topics Curr. Chem. 109(65), 1983; Oppolzer, Tett. Letts., 30:6009, 1989; Helvetica Chimica Acta, 77:2363, 1994; Helvetica Chimica Acta 75:1965, 1992.

More recently, methods for the preparation of backbone labeled, sidechain-unlabeled amino acids have been developed (see U.S. Pat. No. 6,111,066). In these methods, stereo-selective addition of the appropriate amino acid sidechain was added to the isotopically substituted glycine derivatized in a chiral complex.

Therefore, amino acids isotopically substituted (enriched) with ¹³C, ¹⁵N, and ²H or any combination thereof in the backbone of the amino acid residue as below, and that also is isotopically enriched with ²H in the side chain have not been available in the art. Using methods of this invention, such amino acids advantageously may be produced using asymmetric synthesis from glycine, using an appropriately deuterated sidechain precursor. Glycine, specifically labeled with any combination of ¹³C and ¹⁵N, is readily available commercially. Therefore it is preferable to synthesize the amino acids using glycine, isotopically labeled as required, as a precursor. Any other known method may be used to synthesize the desired glycine precursor, labeled in the backbone with any combination of isotopic label(s). The formula below indicates the backbone atoms in bold. R represents the amino acid side chain. Therefore, according to this invention, atoms in the backbone which may be isotopically substituted with any combination of ²H, ¹³C or ¹⁵N are shown in bold below. The alpha-carbon proton is optionally isotopically substituted with deuterium whether the amino hydrogen is substituted with deuterium or not.

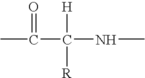

Preferably, however, backbone-labeled glycine first is converted to a nickel II transition metal complex according to the methods of Belokon et al. (J. Chem. Soc. Perkin. Trans. 1:1525-1529, 1992). The derivatized glycine then is alkylated by treatment with a base, such as sodium hydroxide, sodium methoxide or preferably, potassium t-butoxide, followed by addition of the appropriate ²H-labeled sidechain precursor.

Commercially available ²H-labeled sidechain precursors, such as ²H-isopropyl iodide (for valine) or ²H methyl iodide (for alanine) may be used when available, however, not all the sidechain precursors required to produce all twenty naturally-occurring amino acids are available commercially. The present invention therefore provides methods of synthesizing amino acid sidechain precursors or elements thereof in per-deuterated form, allowing any protein or peptide containing any combination of the twenty naturally occurring amino acids to be synthesized in the desired isotopically enriched form.

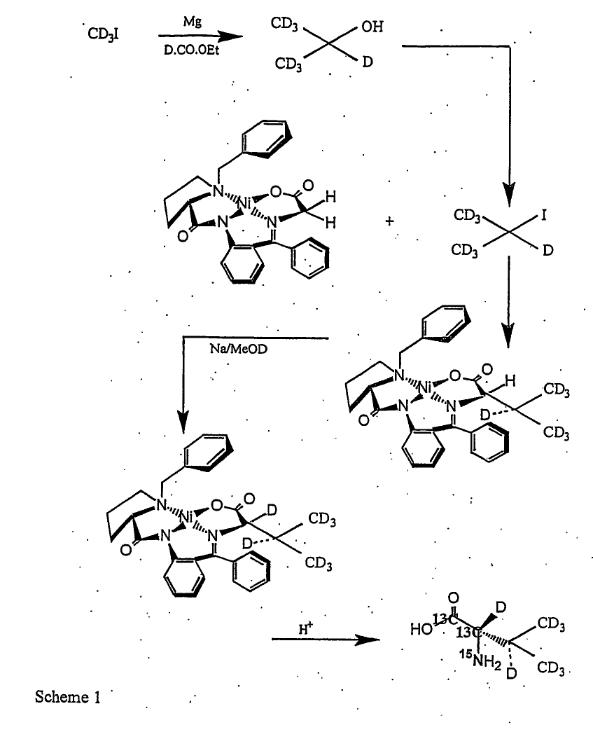

Precursors of this type can be synthesized from commercially available materials. Thus, (CD₃)₂-CD-iodide, the desired precursor for specifically labeled valine, can be prepared from CD₃-labeled methyl iodide via a Grignard reaction with magnesium and deuterated ethyl formate, followed by halogenation of the resulting specifically labeled isopropyl alcohol. The resulting iodide then can be used to synthesize ¹³C, ¹⁵N, and ²H-backbone labeled ²H-sidechain labeled valine. See Scheme 1 (FIG. 1).

Alternatively, deuterated alkyl side chain precursors can be prepared by repeatedly treating unlabeled, water miscible precursors with D₂O in the presence of platinum under high pressure. 2-hydroxy-2-methyl propane is per-deuterated by four treatments with D₂O under these conditions. The perdeutero 2-hydroxy-2-methyl propane then can be converted to the corresponding iodide by treatment with HI, or the corresponding bromide by treatment with phosphorus tribromide. The resulting halide then can be added to the glycine complex in the presence of base to yield protected isoleucine.

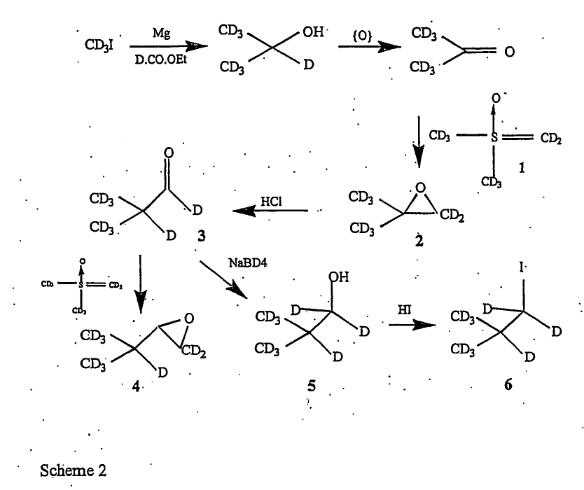

Another suitable method involves assembly of deuterated side chain precursors by successive additions of deuterated methylene groups to a deuterated precursor. Thus, the deuterated side chain precursor for leucine may be assembled as in Scheme 2. See FIG. 2. A deuterated sulfylid (1) is formed by sequentially treating trimethyloxosulfonium iodide with (1) D₂O in the presence of mild base and (2) deuterated DMSO in the presence of strong base such as NaH. The deuterated sulphylid then is added to deuterated acetone to give the epoxy-compound shown as compound 2 in FIG. 2. Rearrangement of the epoxide with acid yields the aldehyde (compound 3). Compound 3 either may be treated with further sulphylid to yield epoxide (compound 4) for further chain extension, or reduced with sodium borodeuteride to give the alcohol (compound 5). Treatment of compound 5 with hydrogen iodide yields per-deutero 1-iodo-2-methyl propane, which on addition to the glycine complex yields protected leucine.

Deuteration at C-α can be achieved by treatment of the alkylated nickel/glycine complex with MeOD in the presence of sodium metal, See FIG. 1. On completion, the deuterated complex is treated with deutero-acetic acid. The desired backbone-labeled, sidechain-deuterated amino acid may be isolated by treatment with aqueous HCl and ion exchange chromatography or by any convenient method known in the art.

It will be apparent to those skilled in the art that these methods may also be employed to synthesize perdeuterated amino acids with no enrichment of ¹³C and ¹⁵N in the backbone by starting with unlabeled glycine. Incorporation of specific backbone-labeled amino acids (for instance, into a binding site) and backbone-unlabeled perdeuterated amino acids (in other locations) into a protein can greatly simplify NMR spectra of peptides and proteins, particularly proteins larger than 20-35 kD. Another embodiment of the present invention therefore provides methods for synthesizing deuterated amino acids that are unlabeled in the backbone.

Methods for incorporating labeled amino acids into proteins designed to avoid scrambling have been described. See Coughlin et al., J. Am. Chem. Soc. 121:11871-11874, 1999 (specifically labeled hCG in mammalian cells) and Giesen et al., J. Biomol. NMR 19:255-260, 2001 (expression of specifically labeled ubiquitin in bacteria). The disclosures with regard to these methods are hereby incorporated by reference in the present specification.

Methods for producing isotopically enriched peptide or protein molecules preferably involve culturing cells that express the molecule in a suitable growth medium that contains at least one isotopically enriched amino acid labeled in the backbone and deuterated in the sidechain as described above. Such molecules may be produced in an isotopically enriched form by culturing cells that express the protein in a suitable growth medium that contains all twenty naturally occurring amino acids (i.e. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine), where all of these amino acids are isotopically enriched or where less than all twenty are isotopically enriched, in a sidechain deuterated form. The medium, as will be appreciated by one of skill in the art, would contain the species of amino acid which are desired to be labeled in the peptidic molecule in an isotopically enriched form while the remaining amino acids would be present in natural abundance form (not enriched with any isotope).

The term “active,” when referring to NMR-active nuclei is used according to the common usage in the art of NMR studies. An active isotope is visible in the corresponding NMR spectrum. Natural abundance refers to the isotopes of an atom that occur in nature. One of skill in the art will recognize that atoms do not exist in a single isotope in nature, and therefore that an atom such as carbon, for example will exist as ¹²C for the most part, but also will exist to a certain degree as ¹³C, naturally. Therefore a carbon-containing molecule that is unlabeled nevertheless will contain a small amount of isotopes other than the natural abundance isotope ¹²C as well. Thus, a carbon position in a molecule that is essentially ¹²C contains ¹²C in the same or essentially the same ratio (abundance) as occurs in nature. Other atoms such as nitrogen and hydrogen also occur naturally as different isotopes and therefore the term “natural abundance” may be understood with respect to any atom. One of skill in the art also will recognize that an isotopically substituted, labeled or enriched atom also is not 100% of the stated isotope but rather is enriched in the stated isotope. The term “enriched” refers to an isotope that is present at greater than natural abundance, up to about 5-100%, usually about 5-20% or about 10-20% and most preferably about 10%. The term “deuterated” refers to isotopic enrichment with deuterium (D or ²H).

Proteins containing specifically labeled amino acids can be chemically synthesized or expressed by bacteria, yeast, mammalian or insect cells or in cell-free systems, as described by Yokoyama et al. The specifically isotopically (enriched) labeled amino acids may be incorporated into cell medium, preferably a mammalian or insect cell medium, individually or in any combination so that the protein expressed by the cells growing in the medium may be specifically enriched with the desired isotopes at the amino acid residues or species of amino acids of choice. The term “species of amino acids” refers to a particular one of the twenty naturally occurring amino acid types. For example, lysine is a species of amino acid as are alanine, glutamic acid and methionine. The term is used to avoid confusion when attempting to distinguish between a single amino acid, i.e. a single residue of a peptidic molecule, as opposed to all instances of a single type of amino acid in the peptidic molecule (one specific alanine in a peptide versus all instances of alanine in a peptide).

Media for bacterial, yeast, mammalian and insect cells (both primary cells and cell lines) are well known in the art. In general, any medium which is sufficient to support the growth of the cells of interest and to support protein expression may be used. Compositions of the type described in U.S. Pat. Nos. 5,324,658; 5,393,669 and 5,627,044 advantageously may be used for the media of this invention, if desired. Likewise, any cell that is capable of expressing the peptidic molecule is suitable for use with this invention. Methods for growing and propagating cells of various types are known in the art. Any suitable method in which the cells can express the isotopically enriched protein may be used with the methods and compositions of this invention. Culture conditions in which the protein of interest is expressed in quantities sufficient to isolate the material from the cell culture or medium are termed “protein-producing conditions.”

Persons of skill in the art are aware of many methods for isolating proteins and peptides from cells or from cell media. Any of these methods may be used according to this invention. Peptidic molecules, once isolated in isotopically enriched form can be studied according to known methods. Any method for subjecting the proteins to nuclear magnetic resonance study is contemplated for use with the methods and compositions of the invention, but preferably multidimensional NMR methods such as TROSY, HNCA, HNCOCA, HNCO and HNCACO are employed.

### EXAMPLES

### Example 1

### Synthesis of L-(¹³C₂, ¹⁵N, 50%-²H-Backbone)-Sidechain-U—²H Valine

BPB—Ni (II)-(¹³C₂, ¹⁵N)-Glycine red complex (1 g, 0.97 mmol, 1.00 equiv.) was suspended in anhydrous CH₃CN (20 mL) at room temperature. NaO^{t}Bu (0.2 g, 2.1 mmol, 1.05 equiv.) was added to the red reaction suspension followed after 5 minutes by (CD₃).CD-iodide (0.21 ml, 2.1 mmol, 1.05 equiv.) in anhydrous CH₃CN (10 mL). After 4 hours, thin layer chromatography (silica gel, acetone/CHCl₃=1/5) revealed the presence of a trace of unreacted starting material and a major spot with a higher R_{f} value. Glacial acetic acid (0.24 mL, d=1.049, 63.95 mmol, 4.41 equiv.) was added to quench the reaction.

The reaction mixture was concentrated under reduced pressure and extracted with CH₂C1₂ (3×50 mL). The combined organic layers were washed with H₂O (2×50 mL) and then brine solution (50 mL). The organic phase was dried (MgSO₄) and evaporated to provide a red crude foamy glass. The crude product was subjected to further purification by flash column chromatography on silica gel using chloroform:acetone as eluant. The approporiate fractions were combined and evaporated to dryness to provide BPB—Ni(II)-(¹³C₂, ¹⁵N—¹H-backbone)-sidechain-U—²H valine.

One half of the residue was dissolved in MeOD (Isotec, 26 mL), treated with sodium metal (92 mg) and the whole heated to reflux overnight. On cooling, the reaction mixture was treated with deutero-acetic acid (CIL, 1.4 ml) and concentrated under reduced pressure. The mixture was extracted with CH₂C1₂ (3×50 mL) and the combined organic layers were washed with H₂O (2×50 mL) and then brine solution (20 mL). The organic phase was dried (MgSO₄) and evaporated. The resulting red foamy glass was dissolved in methylene chloride (10 ml) and added dropwise to stirred hexane (2 L). The suspension was stirred overnight, filtered and the collected solid dried to provide BPB—Ni (II)-(¹³C₂, ¹⁵N, —²H-backbone)-sidechain-U—²H valine.

A 1:1 mixture of BPB—Ni (II)-(¹³C₂, ¹⁵N, —¹H-backbone)-sidechain-U—²H valine and BPB—Ni (II)-(¹³C₂, ¹⁵N, —²H-backbone)-sidechain-U—²H valine, CH₃OH (60 mL), in 2 M HCl (60 mL) were heated at reflux for 10 minutes. The pale green solution was evaporated to dryness on a rotary evaporator. H₂O (50 mL) was added to the dried solid. The mixture was cooled in an ice bath for several hours. Filtration of the mixture gave BPB.HCl. The filtrate was dried and the title compound isolated by ion exchange chromatography and crystallized from aqueous ethanol as (¹³C₂, ¹⁵N, 50%-²H-backbone)-sidechain-U—²H valine. (M/S contains molecular ions at m/z 128 and 129).

### Example 2

### Synthesis of perdeutero-1-iodo-2-methylpropane

Trimethyloxosulfonium iodide (110 g, 0.5 mmol) was dissolved in hot D₂O (500 ml). Potassium carbonate was added and the solution heated to 70-90° C. for one hour, then cooled to approximately 0° C. for one to two days. The resulting solid was filtered and the process repeated twice to yield perdeutero-trimethyloxosulfonium iodide (yield, 76.5 g, 66.8%. M/S contains molecular ion at m/z 102).

Sodium hydride (6 g) was placed in a 500 mL flask and washed with petroleum ether by stirring and decanting. Residual ether was removed under reduced pressure. d6-dimethyl sulfoxide (150 mL) was added and the suspension heated to 60-70° C. until effervescence had ceased. The mixture was cooled with cold H₂O. Perdeutero-trimethyloxosulfonium iodide (57.29 g, 250 mmol) was added and the mixture stirred for 15 minutes. d6-acetone (12.8 mL, 200 mmol) then was added. The mixture was stirred at room temperature for 30 minutes and then heated to 40-45° C. for 30 minutes. After cooling and stirring at room temperature for a further hour, the flask was fitted with a distillation adapter, condenser and a receiver flask cooled to −70° C. The system was placed under water aspirator vacuum and the reaction flask heated to 50° C. to isolate perdeutero-methylpropylene oxide (11.7 g, 75%).

Perdeutero-methylpropylene oxide (16.44 g, 205 mmol) was cooled in an ice bath and treated with DCl in D₂O (100 ml) and the whole refluxed for 18 hours. The reflux condenser was replaced by a distillation apparatus and perdeutero-isobutyraldehyde isolated by distillation. Yield, 9.65 g (58%).

Perdeutero-isobutyraldehyde (9.65 g, 120 mmol) was suspended in D₂O and cooled in an ice bath. Sodium borodeuteride (5 g, 120 mmol) was added in portions over a 10 minute period. The mixture was stirred for 1 hour and then sodium chloride (approximately 12 g) was added. The mixture was extracted with diethyl ether (4×50 mL) and the organic extracts dried (sodium sulfate) and distilled through a column containing glass helices to give perdeutero-isobutyl alcohol as colorless liquid (bp 105-108° C.; yield, 8.7 g (88%).

Perdeutero-isobutyl alcohol (8.7 g, 105 mmol) was stirred in an ice bath while hydroiodic acid (50 mL) was slowly added. The mixture was then heated in an oil bath and slowly distilled. Crude perdeutero-isobutyl iodide was isolated (bp 80-98° C.). Water was removed with a burette and treatment with sodium sulfate, followed by filtration. Color was removed by treatment with sodium metabisulfite and filtration to yield pure per deutero-1-iodo-2-methylpropane.

### Example 3

### Expression and Purification of Serine Hydroxymethyl transferase (SHMT) Containing Backbone-¹³C₂, ¹⁵N, ²H-Sidechain ²H₇-L-valine

A 20 mL stock culture of M15 cells transformed with the vector pqe30 SHMT was used to inoculate 1 L of medium containing 500 mg alanine, 400 mg arginine, 400 mg aspartic acid, 50 mg cysteine, 400 mg glutamine, 650 mg glutamic acid, 550 mg glycine, 100 mg histidine, 230 mg isoleucine, 230 mg leucine, 420 mg lysine HCl, 250 mg methionine, 130 mg phenylalanine, 100 mg proline, 2.1 g serine, 230 mg threonine, 170 mg tyrosine, 230 mg valine, 500 mg adenine, 650 mg guanosine, 200 mg thymine, 500 mg uracil, 200 mg cytosine, 1.5 g sodium acetate (anhydrous), 1.5 g succinic acid, 750 mg NH₄Cl, 850 mg NaOH, 10.5 g K₂HPO₄ (anhydrous), 2 mg CaCl₂ 2H₂O, 2 mg ZnSO₄ 7H₂O, 2 mg MnSO₄ H₂O, 50 mg tryptophan, 50 mg thiamine, 50 mg niacin, 1 mg biotin, 20 g glucose, 4 mL 1 M MgSO₄, 1 mL 0.01 M FeCl₃, 15 mg ampicillin, and 50 mg kanamycin.

When cell density had reached an OD of 1.2, the cells were harvested by centrifugation, rinsed with PBS, recentrifuged and resuspended in a medium of the above proportions but in which backbone-¹³C₂, ¹⁵N, ²H,-sidechain-²H₇-L-valine ²H₇-L-valine was substituted for the unlabeled valine. After 30 minutes, protein expression was induced by addition of IPTG to a final concentration of 0.1 mmol. After 6 hours, the cultured cells were centrifuged at 4000 rpm for 20 minutes in a Sorvall RC-3B centrifuge. The cell pellet was then stored at −20° C. overnight. The cells were thawed and resuspended in 30 ml of sonication buffer (50 mM sodium phosphate, 500 mM NaCl, pH=8.0). The cells were broken by passing them through a French Press four times at 20,000 psi. The broken cells were subjected to sedimentation at 15,000×g for 20 minutes in Oakridge tubes.

A 5 ml Ni-NTA immobilized metal affinity column was equilibrated in sonication buffer at 5 mL/min. The supernatant (cell lysate) was removed from the Oakridge tube without disturbing the pellet. Cleared lysate was loaded onto the column at 5 mL/min. The column flow-through was saved for later analysis. The material bound to the column then was washed with sonication buffer for 30 minutes until the absorbence of the effluent was less than 0.020. Bound protein was eluted from the column with Elution Buffer (50 mM sodium phosphate, 500 mM NaCl, 500 mM imidizole, pH=8.0) using a single step. The peak fraction was collected manually. A sample of the elution was saved for analysis.

A 300 mL XK-50 column packed with Sephadex G-25 Fine size exclusion chromatography (SEC) resin was equilibrated with anion exchange Buffer A (20 mM Tris HCl, pH=7.5). The Ni-NTA eluate was loaded onto the SEC column at 15 mL/min. The protein peak was collected manually. The protein sample, now in anion exchange Buffer A, was stored at 4° C. during preparation of the next step. A 10 mL Resource Q anion exchange column was equilibrated in Buffer A. The partially purified protein was loaded onto the column at 10 mL/min. The sample was washed with Buffer A for three minutes. The sample was eluted with a linear gradient into Buffer B (20 mM Tris HCl, 1 M NaCl, pH=7.5) over seven minutes. The fractions were collected in 30 second intervals. A sample of each fraction was set aside for analysis.

The material was analyzed by SDS-PAGE using a 12% Tris/Glycine gel at a constant 200 volts for 45 minutes. The pure fractions were loaded into a 3000 MWCO Slidalyzer™ dialysis cassette. The protein was dialyzed at 4° C. into 50 mM sodium phosphate pH 7.0. Two buffer changes ensured complete removal of the Tris buffer. The final protein concentration was determined using UV absorbance at 280 nm; comparing it to the extinction coefficient for MUP (0.503 at 1 mg/mL). The final concentration of pure Serine Hydroxymethyl Transferase was 48 mg/mL in 1.9 mL. Total yield was 90 mg. The final SHMT sample was stored at 4°C. prior to NMR analysis.

### Example 4

### NMR Analysis of Serine Hydroxymethyl Transferase SHMT) Containing Backbone-¹³C₂, ¹⁵N, ²H-Sidechain ²H₇-L-Valine

A 15 mg sample of backbone-¹³C₂, ¹⁵N, ²H,-sidechain-²H₇-L-valine labeled MUP was dissolved in 650 μL phosphate buffered saline (10 mM potassium phosphate; 200 mM sodium chloride), to which was added 50 μL deuterium oxide. A three-dimensional HNCA spectrum was acquired according to known methods.

1. An amino acid wherein the sidechain of said amino acid is isotopically enriched with ²H and wherein the backbone of said amino acid is isotopically enriched with an isotope selected from the group consisting of ¹³C, ¹⁵N, ²H and any combination thereof, with the proviso that said amino acid is not isotopically enriched with ²H at every hydrogen.

2. An amino acid of claim 1, wherein the backbone of said amino acid is isotopically enriched with an isotope selected from the group consisting of ¹³C, ¹⁵N, ²H and any combination thereof.

3. An amino acid of claim 1, wherein the α-carbon proton of said amino acid is isotopically enriched with ²H.

4. A method of synthesizing the amino acid of claim 1, which comprises:
(a) obtaining glycine that optionally is isotopically enriched in the backbone with an isotope selected from the group consisting of ¹³C, ¹⁵N and ²H or any combination thereof;
(b) chemically derivatizing said glycine;
(c) adding a deuterated side chain to said chemically derivatized glycine in a stereo-selective manner to produce a protected sidechain deuterated amino acid; and
(d) deprotecting said sidechain deuterated amino acid.

5. A method of synthesizing the amino acid of claim 2, which comprises:
(a) obtaining glycine that optionally is isotopically enriched in the backbone with an isotope selected from the group consisting of ¹³C, ¹⁵N and ²H or any combination thereof;
(b) chemically derivatizing said glycine;
(c) adding a deuterated side chain to said chemically derivatized glycine in a stereo-selective manner to produce a protected sidechain deuterated amino acid;
(d) deuterating the α-carbon of said protected sidechain deuterated amino acid; and
(e) deprotecting said sidechain deuterated amino acid.

6. A peptidic molecule which comprises at least one amino acid of claim 1.

7. A peptide molecule which comprises at least one amino acid of claim 2.

8. A peptide molecule which comprises at least one amino acid of claim 3.

9. A peptide molecule which comprises at least one species of amino acid wherein the side chain of each occurrence of said species of amino acid is isotopically enriched with ²H.

10. A peptide molecule of claim 9, wherein the backbone of each occurrence of said species of amino acid is isotopically enriched with an isotope selected from the group consisting of ¹³C, ¹⁵N, ²H and any combination thereof.

11. A peptide molecule of claim 9, wherein the α-carbon proton of each occurrence of said species of amino acid is isotopically enriched with ²H.

12. A medium capable of supporting the growth of cells in culture which comprises at least one amino acid of claim 1.

13. A medium capable of supporting the growth of cells in culture which comprises at least one amino acid of claim 2.

14. A medium capable of supporting the growth of cells in culture which comprises at least one amino acid of claim 3.

15. A method of producing an isotopically labeled peptide molecule, which comprises:
(a) providing a medium of claim 12;
(b) providing a cell culture that expresses said peptide molecule;
(c) growing said cell culture in said medium under protein-producing conditions such that said cell expresses said peptide molecule in isotopically labeled form; and
(d) isolating said isotopically labeled peptide molecule from said medium.

16. A method of producing on isotopically labeled peptide molecule, which comprises:
(a) providing a medium of claim 13;
(b) providing a cell culture that expresses said peptide molecule;
(c) growing said cell culture in said medium under protein-producing conditions such that said cell expresses said peptide molecule in isotopically labeled form; and
(d) isolating said isotopically labeled peptide molecule from said medium.

17. A method of producing on isotopically labeled peptide molecule, which comprises:
(a) providing a medium of claim 14;
(b) providing a cell culture that expresses said peptide molecule;
(c) growing said cell culture in said medium under protein-producing conditions such that said cell expresses said peptide molecule in isotopically labeled form; and
(d) isolating said isotopically labeled peptide molecule from said medium.

18. A method of determining structural information for a peptidic molecule, which comprises:
(a) producing said peptidic molecule according to the method of claim 15; and
(b) subjecting said peptidic molecule to nuclear magnetic resonance.

19. A method of determining structural information for a peptidic molecule, which comprises:
(a) producing said peptidic molecule according to the method of claim 16; and
(b) subjecting said peptidic molecule to nuclear magnetic resonance.

20. A method of determining structural information for a peptidic molecule, which comprises:
(a) producing said peptidic molecule according to the method of claim 17; and
(b) subjecting said peptidic molecule to nuclear magnetic resonance.
